(19) United States
(12) Patent Application Publication
(10) Pub. No.: US 2022/0062456 A1
(43) Pub. Date: Mar. 3, 2022

(54) Non-Contact Ultrasound Disinfection System and Method

(71) Applicant: Anarkali Mahmood, Denver, CO (US)

(72) Inventor: Anarkali Mahmood, Denver, CO (US)

(21) Appl. No.: 17/410,813

(22) Filed: Aug. 24, 2021

Related U.S. Application Data
(60) Provisional application No. 63/070,075, filed on Aug. 25, 2020.

(51) Int. Cl. A61L 2/025

(52) U.S. Cl. CPC A61L 2/025; A61L 2202/14; A61L 2202/12; A61L 2202/20

(57) ABSTRACT

A system and method for disinfecting a person's skin and clothing, using non-contact ultrasonic waves is disclosed. This involves a ring or other assembly that includes a number of ultrasound transducers that surround a person, and that direct ultrasonic waves inward towards the person. The transducer assembly may be moved along the person's height so that the person's skin and clothing are disinfected.

### CROSS REFERENCE TO RELATED APPLICATION

This application claims the benefit of U.S. Provisional Application No. 63/070,075, filed Aug. 25, 2020, the contents of which are incorporated herein by reference.

### FIELD OF THE INVENTION

The field of the current invention regards disinfection of harmful pathogens, such as the coronavirus, including the disinfection or inactivation of pathogens present on a person's skin or clothing using non-contact ultrasound, i.e., ultrasonic waves transmitted through the air.

### BACKGROUND OF THE INVENTION

Highly infectious pathogens such as SARS-CoV2, Ebola, and H1N1 are one of the biggest threats to human health due to their exponential growth rates and their ability to cause life-threatening diseases. The rapid spread of SARS-CoV2 and the resulting pandemic have reminded us of the devastating results of such pathogens and have highlighted the need for technologies that can hinder and/or limit the spread of such viruses into and within our public spaces. The current pandemic necessitates that we install and implement every possible sanitary checkpoint to prevent the transmission of such pathogens.

Pathogen spread can occur via numerous direct and indirect routes, however, such as pathogenic spread via contaminated fomites (i.e. clothing) and/or exposed human body. In hospitals and laboratories, pathogen transmission via contaminated fomites is usually controlled by the use of lab coats; however, lab coats have to be utilized appropriately, as their improper use can make them a source of pathogenic spread themselves, rendering them ineffective. Additionally, the use of lab coats is not practiced by the rest of public and requiring people to wear lab coats might not only cause unnecessary panic and interruptions to everyday life, but will also result in increased biohazardous waste that is toxic to both humans and the environment. Thus, there is a need for a sanitization system and method that can hinder the transfer of pathogens traveling via contaminated human clothing and/or skin.

Currently available disinfection techniques have several inherent limitations that make them inapplicable for safe, real-time disinfection of human skin or clothing. For example, current disinfection methods involving the use of aqueous chemicals, such as chlorine (e.g. chlorine dioxide or bleach), alcohol, and ethylene oxide, not only require wetting the clothing items being treated but also require exposure to highly toxic chemicals. The adverse health risks posed by soaking items/skin with dangerous chemicals makes them unsuitable for applications involving human exposure to the disinfectant.

Disinfection without exposure to aqueous chemicals can be achieved using ultraviolet light, gamma radiation and water-coupled high frequency ultrasonic waves. However, ultraviolet light and gamma radiation based techniques involve exposure to carcinogenic radiation and cannot be utilized for purposes where human exposure to disinfectant is required. The adverse health effects posed by radiation exposure outweighs any potential benefit of disinfection achieved via their application. Additionally, the current water-coupled ultrasound disinfection methods not only involve submerging a contaminated item in water, as the ultrasonic waves require water to reach the item, but also use high frequency ultrasonic waves which may be dangerous for human beings. Therefore, ultrasound disinfection, in its current form cannot be utilized for real-time disinfection of human clothing or skin.

Low frequency ultrasound based diagnostic and therapeutic techniques are widely employed in the medical field, as human exposure to low frequency ultrasound waves is generally considered to be safe. These techniques require transmission of ultrasonic energy into the body. Since ultrasonic beams cannot penetrate human skin if traveling via air, such techniques not only require physical contact between a patient's skin and the source of ultrasound waves (i.e. transducer) but also require application of a conductive medium (e.g. gel) to eliminate any air space between the skin and the transducer. Given the disinfection potential of ultrasonic waves and their well-established safety for human exposure, one potential disinfection system that can be developed to deliver safe and effective disinfection in real-time involves the use of air-coupled, low frequency ultrasound waves as the disinfectant.

As existing disinfection systems use harmful radiation, extremely high temperatures, harsh chemicals, or have other drawbacks, the need for disinfection systems and methods that are safer for use with human remains. Specifically, we need a sanitization system and method that is both safe for humans and can effectively eliminate and/or deactivate pathogens such as viruses. Thus there is a need for a disinfection system and method that involves the benefits of safe ultrasonic waves, and delivers ultrasonic waves without an aqueous coupling media.

### SUMMARY OF THE INVENTION

The current invention is described in the Detailed Description of the Preferred Embodiments, as well as in the claims. Among other inventive aspects, the current invention involves the following.

1. Use of non-contact ultrasound traveling through the air to disinfect or sterilize a person's skin or clothing.

2. Use of ultrasonic waves at an intensity and duration that results in the inactivation of harmful pathogens existing on a person's skin or clothing, but not significantly harming the helpful microbes also existing on a person's skin or clothing that help fight the spread of the harmful pathogens.

3. A high-throughput ultrasound based system that may efficiently inactivate pathogens traveling via fomites so that it can be used in a busy location.

4. A system that includes a number of ultrasound transducers that surround the person using the system, where the ultrasound transducers are moved up or down along the person's height to inactivate pathogens present on that person's skin and clothing.

5. A system where a number of transducers are mounted to a ring and emit ultrasonic waves inward towards a person standing within the ring. As ultrasonic waves are emitted inward towards the person in the ring, the ring may simultaneously move up or down along the person's height to inactivate pathogens present on that person's skin and clothing using ultrasonic energy.

6. A system where the ultrasound intensity, exposure time and distances between the ultrasound transducers and person using the system, may be varied to achieve different levels of disinfection.

7. Additionally, the system may be equipped with a source of air supply and with air nozzles (present) on the ring mount to provide air or some other fluid, e.g., high velocity air, traveling in the same or similar direction as the ultrasonic waves. It is preferred that by coupling high velocity air with ultrasonic waves, the air may provide a medium for the ultrasonic waves to travel in facilitating delivery of the waves at the desired intensity and distance, i.e., intensity and distance required to reach a potential source of contamination and disinfect it.

It is an object of the present invention to provide high-throughput chemical-free systems and methods to lessen and/or eliminate contaminants traveling via fomites or exposed skin at entrances to public spaces. The object of the present invention can be obtained via embodiments listed below or via combination of the various embodiments or via combination of different components of the embodiments listed below.

Other aspects of the present invention are described herein.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 shows an ultrasound disinfection system.

FIG. 2 shows an ultrasound disinfection system.

FIG.3 shows an ultrasound source in proximity to a person.

FIG. 4 shows a mount for positioning ultrasound sources.

FIG. 5 shows an ultrasound disinfection system

FIG. 6 shows a mount for positioning ultrasound sources and air flow sources.

FIG. 7 shows an ultrasound disinfection system.

FIG. 8 shows an ultrasound disinfection system.

FIG. 9 shows an ultrasound disinfection system.

### DETAILED DESCRIPTION OF THE PREFERRED EMBODIMENTS

The current invention generally involves the use of non-contact ultrasonic waves to disinfect pathogens on a person's skin or clothing in an efficient manner. The non-contact ultrasound disinfection system and method of the current invention overcome many disadvantages of current disinfection methods. For example, the non-contact ultrasound utilized in the current invention is safe for humans, and thus allows decontamination of a person's skin as well as that person's clothing, without causing any harm to the person or clothing material.

In contrast to current disinfection methods, non-contact ultrasonic disinfection may provide comparable yet safer disinfection of pathogens without exposure to potentially carcinogenic radiation (e.g., during use of ultraviolet light) or to harmful chemicals (such as chlorine, alcohol, and ethylene oxide). To this end, an advantage of the present invention is that non-contact ultrasound may achieve as high as 99.9% of bacterial inactivation. Such microbes are a source of infectious pathogens which can be transmitted to other people utilizing the same public space.

The use of non-contact ultrasound in the present invention also differs from existing uses of ultrasound to assess or treat locations within a person (in medical diagnostic and therapeutic techniques) or an item (during product inspection). That is, the use of non-contact ultrasound in the current invention does not require that the ultrasound source(s) be in contact with the person's skin or clothing.

Indeed, the current invention has recognized that pathogens on a person's skin or clothing, i.e., on or near the surface of the person's skin or clothing, may still be inactivated with non-contact ultrasound. That is, the current invention has discovered that any attenuation occurring through the air before contacting the person's skin or his/her clothing, is not so significant as to render the ultrasonic waves ineffective in inactivating pathogens on or near the surface of the person's skin or clothing. This is in sharp contrast to existing systems where the location of interest is within a person or item; where the attenuation through air renders it ineffective by the time the ultrasound penetrates the person or item and reaches the location of interest.

It should be noted that, while the following description generally refers to “disinfection” when describing the process of inactivation or reduction of pathogens, the scope of the current invention also covers both decontamination and sterilization processes; where “decontamination” may generally refer to the process of destroying, inactivating, neutralizing and/or killing most pathogens in the contaminated item; and where “sterilization” may generally refer to the process of destroying, inactivating, neutralizing and/or killing all pathogens in the contaminated item. As such, the use of any of the terms “disinfection,” “decontamination” or “sterilization” in this application is not intended to exclude the other process terms, nor limit the type of process provided by the current invention, unless where specifically intended or described. And the current invention may provide different processes to accommodate different desired levels of pathogen removal or inactivation. As such, the following description may include instances where a certain level or levels of pathogen removal or inactivation occur. Furthermore, use of the term “pathogen” herein generally refers to various contaminants or infectious agents, such as bacterium, viruses, such as the coronavirus, prions, spores and other contaminants. As such, the scope of the current invention covers the decontamination and/or sterilization of a wide array of pathogens.

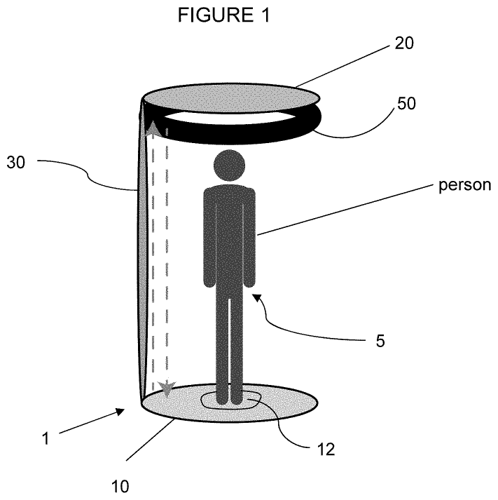

Disinfection system 1 and associated methods according to preferred embodiments of the current invention are now described with reference to the figures. It should be noted that disinfection system 1 shown in FIGS. 1-4, or the disinfection systems shown in the other figures, are for example purposes only, and the current invention is not limited to the configurations shown. Instead, and as discussed herein, system 1 may comprise different configurations to best suit the application for which the system is used, where it is located and/or the logistics factors it is intended to meet.

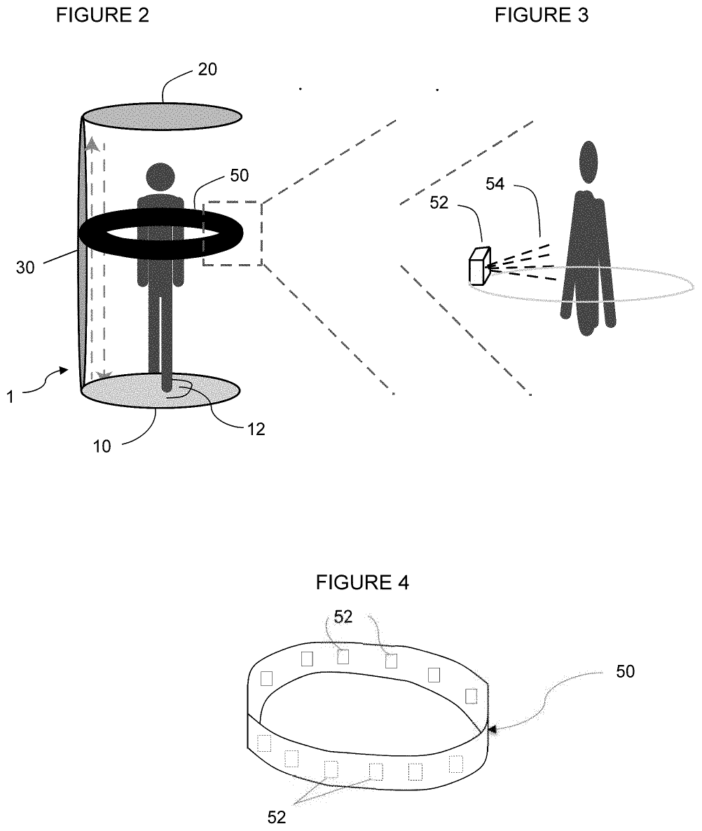

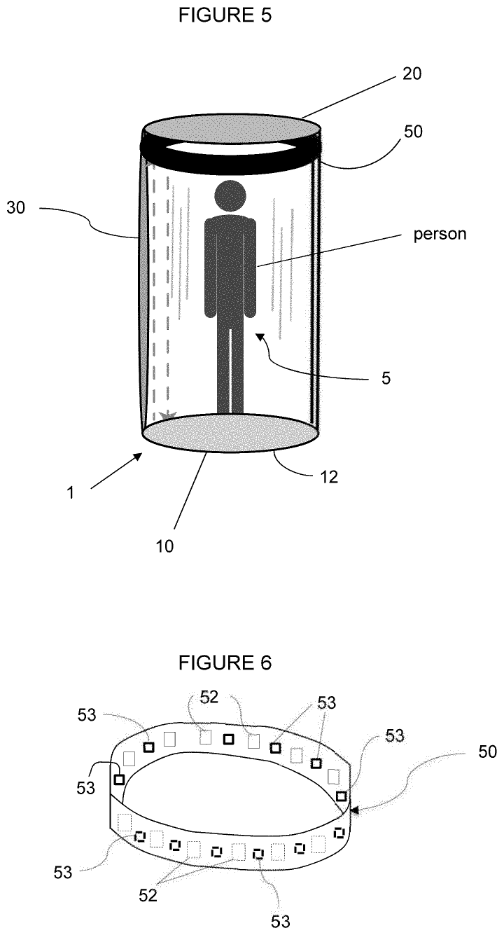

As shown in FIGS. 1-2, system 1 may include disinfection, decontamination or sterilization space, zone or region 5 in which a person may stand or otherwise be located, and in which disinfection, decontamination or sterilization may occur. As noted above, system 1 may be used for disinfection, decontamination or sterilization, but the following description generally references system 1 in connection with disinfection. Disinfection space 5 may generally be formed or defined by components of system 1, which may include base 10, top 20, one or more strut(s) or vertical member(s) 30, and/or ultrasound source assembly 50. In general, a person may stand within system 1, and ultrasound assembly 50 may emit ultrasonic waves inward towards the person, as assembly 50 moves vertically along the person's height, thereby disinfecting the person's skin and clothing.

The structural components of system 1 may comprise aluminum, steel or other suitable materials that provide sufficient strength to accommodate a person's weight and/or the forces created as the ultrasound assembly 50 moves up or down. To this end, as shown in FIG. 6, multiple vertical struts or members 30 may be located about the circumference or periphery of base 10 and top 20 to provide structural integrity. As explained later, one or more struts 30 may support movable ultrasound assembly 50. The configuration of system 1 may be cylindrical but other cross-sectional configurations may be used, e.g., elliptical, square, rectangular or other configuration.

The structure of system 1 is preferably sized so that disinfection space 5, where the person stands during disinfection, is tall and wide enough to accommodate many or most peoples' heights and weights. For example, the structure's height may be about 82″ and its diameter may be about 54″. However, other dimensions may be used, and the foregoing dimensions are only for example purposes and do not limit the scope of the invention. Furthermore, system 1 may be configured to accommodate people in a sitting position, such as a person in a wheelchair. In this situation, the person's skin, clothing and wheelchair surfaces may be disinfected.

Disinfection space 5 may be enclosed to relatively contain the ultrasonic waves and/or generally isolate the disinfection process from the surrounding area. To this end, the system may include a tubular or other shaped enclosure 70. The enclosure may be transparent, and may comprise plexiglass or other suitable material. Enclosure 70 may also add to the structural integrity of system 1, and may be coupled to base 10 and/or top 20, as well as strut(s) 30. Enclosure 70 may include a door (not shown) or an opening that becomes accessible to a person as enclosure 70 rotates, but that seals disinfection space 5 by further rotation before disinfection begins. As an alternative, disinfection space 5 need not be enclosed.

System 1 may also include other structural members such as horizontal rings (not shown) that may be coupled to strut(s) 30 at different heights, to provide additional structural integrity. Such horizontal rings may also be positioned at the bottom and/or top of system 1, and may help couple base 10 and top 20 to the rest of system 1.

As explained in more detail later, base 10 may form the location on which the person stands or is otherwise located. Base 10 may include an interior or active region 12 which may itself include one or more ultrasound sources to emit ultrasonic waves upward. In this manner, the soles of the person's shoes or exposed skin of the person's feet, ankles and lower shins may be disinfected. Base 10 may also include rollers or wheels (not shown), which may allow system 1 to be moved to different locations. Such rollers or wheels may be locked in position when system 1 is positioned at the desired location.

System 1 may also be tilted and moved to another location. In this embodiment, base 10 may comprise a square or rectangular (or other non-circular) shape to provide stability when system 1 is tilted and moved. Strut(s) 30, or enclosure 70 may include handles (not shown) to facilitate tilting and/or moving system 1.

Top 20 may be included to provide structural integrity to system 1, which may help make system 1 portable. Where system 1 is located outside, it is preferred that it include top 20 to protect persons being disinfected from rain or the elements.

As indicated above, ultrasonic waves are generally harmless, but it may still be preferable for the next persons to be disinfected to wait a certain distance away from system 1. Generally, ultrasound transducers may have an effective range through air up to a distance, so in one example, the next person to be disinfected may stand an appropriate distance away from system 1 so as to not receive multiple doses of ultrasonic waves while waiting. But in keeping with existing social distancing parameters, the next person may in any event stand six feet or more while waiting to be disinfected.

Ultrasound source assembly 50 is now further described with reference to FIGS. 1-4. Assembly 50 may comprise a ring mount, or other type of support structure, that may be lowered and/or raised along the person's height during a disinfection process. Ring 50 may include or support an ultrasonic wave generator 52, or a number of ultrasonic wave generators (i.e., transducers) 52, that are spaced about the ring's circumference. Transducers 52 may direct ultrasonic waves 54 inward, to reach the exposed skin/clothes of a person positioned within system 1 (or within designated space 5) as the ring 50 travels along the person's height. It is preferred to use a number of ultrasound transducers 52 that are positioned around the person so that the ultrasonic waves may reach any pathogens from his or her front, rear, sides, etc. In general, ultrasound sources 50 are located in a non-contact position in proximity to the person, i.e., at a location and distance from the person so that the disinfection procedure achieves the desired level of disinfection.

Ultrasound assembly 50 may be formed as a ring or circle, but alternatively as an ellipse. An ellipse assembly 50 may be preferred because it generally corresponds to the cross-sectional shape of a person. This may result in a more uniform ultrasound dosage to different areas of the person's skin or clothing because the ultrasonic waves may travel approximately the same distance through air before encountering the person. However, assembly 50 may comprise different configurations such as a square, rectangle or other shapes.

Ultrasound assembly 50 may be coupled to a motor or other power device (not shown) that serves to move ring mount 50 (and thus the ultrasound sources 52) along the person's height, preferably vertically, simultaneously as ultrasonic waves 54 are generated and directed towards the person. For example, ring mount 50 may be coupled to a jack screw (not shown) that is in turn powered by a motor. The motor may turn the jack screw, which in turn may lower or raise ring mount 50 when the person is scanned or exposed to ultrasonic waves during disinfection. Alternatively, the motor may be directly attached to ring mount 50 to provide movement. Alternatively, the motor may drive a pulley and cable system (not shown) that is coupled to assembly 50 to provide vertical or other movement.

Regardless of how the motor, or other drive component that moves assembly 50, is configured, the drive component may provide, for example, linear vertical movement of ring mount 50 at about two (2) inches per second. However, other scan speeds may be used, which may depend on the desired dosage to be delivered to the person, and the foregoing parameters are for example purposes only and do not limit the scope of the invention. The scan speed, and the corresponding dosage delivered to the person may depend on, for example, whether the person has hair on the arms and legs. To this end, men with hairy arms may warrant a larger dosage. The scan speed, and the corresponding dosage delivered to the person may also depend on the type of clothing worn by the person. For example, clothing that is relatively flat and uniform, e.g., yoga pants, may involve a smaller dosage than, e.g., baggy clothing. In any event, it should be noted that the invention includes any variable of scan speed in the vertical (Z) or other direction. The scan speed and dosage may be controlled by software to result in the desired level of disinfection, decontamination or sterilization.

Ring mount 50 may have a diameter of approximately 50″, which may accommodate the physical dimensions of many or most people. However, other ring mount diameters may be used, and the foregoing dimension is for example purposes only and does not limit the scope of the invention. (And as noted above, mount or assembly 50 may be configured as an ellipse or other shape.) The vertical and radial dimensions of ring mount 50 are preferably large enough to house a number of ultrasound transducers 52. For example, ring mount 50 may include approximately 176 transducers 52, each having a diameter of about 2.5″ and horizontal spacing of approximately 0.5″. Again, the foregoing dimensions and parameters are for example purposes only and do not limit the scope of the invention.

In this embodiment, ultrasonic transducers 50 are capable of generating ultrasonic waves 54 in a frequency range of 30 kHz to 170 kHz, based on an input voltage of between about 50 V to about 100 V. Ultrasonic transducers 50 may continuously produce sonic waves during a disinfection process to inactivate aerosolized virus particles on the person's skin or clothing at a distance between 16″ to 19″, with a step size of 10″, where transducers 50 move at a linear speed of about 2″/sec. It is believed that this combination of parameters, i.e., dimensions, ultrasound intensity and exposure duration, may provide sufficient ultrasonic energy to effectively disinfect or sterilize the skin and clothing of many or most persons. However, as noted above, the scope of the invention is not limited to these parameters, and the frequency or frequency range, input voltage or voltage range, distance, step size and scan speed may vary.

However, these parameters may be varied to accommodate higher levels of disinfection. For example, the linear speed of the ring mount and its ultrasound transducers may be slowed to 1″/sec thereby increasing the exposure duration, and may result in a generally higher level of inactivation of pathogens, e.g., may achieve sterilization. Other parameters may be varied as well, and software may be used to control various aspects of system 1 so that as parameters are varied, the desired level of inactivation of pathogens is preferably achieved.

It is preferred that assembly 50 may accommodate any wiring necessary to power transducers 52. For example, ring or ellipse 50 may be hollow to allow wiring to extend therein. Such wiring may conclude at a bus (not shown) that may then connect to a cable assembly (not shown) outside of assembly 50 where the cable assembly has sufficient length to allow movement of assembly 50 along the length of the person's height. In addition, or as an alternative, aspects of system 1 may be controlled wirelessly.

Most pandemics result from uncontrollable spread of infectious viruses and thus can be controlled by limiting the spread of such infectious viruses. The first line of defense against harmful microbes consists of trillions of helpful microbes (i.e., bacteria, viruses and fungi) present on our skin. However, the helpful microbes are not sufficient during an outbreak of infectious diseases and require the use of disinfectants for disease prevention.

However, current disinfectants though potent, generally act as blunt instruments. That is, current disinfectants may not only destroy harmful pathogens, but also result in the inactivation of the helpful microbes. The current invention addresses this situation because it is tuned to specifically target infectious viruses with limited harm to helpful microorganisms. Ultrasonic disinfection may be used to achieve targeted viral inactivation by altering ultrasound intensity, exposure time and distance. And while different levels of disinfection are referenced herein, it may be desirable to not disinfect to a higher level, e.g., sterilization, if that would adversely impact the helpful microorganisms located in the area to be addressed.

Viruses, especially lipid encapsulated viruses such as SARS COV2 are generally easier to inactivate. This means that highly infectious viruses like SARS COV2 require a low level disinfection. To achieve low level disinfection for pathogen inactivation, ultrasonic waves may be generated within a frequency range between about 30 kHz to about 100 kHz at an exposure rate of 2 inches/sec. Other ultrasound dosages may be generated to inactivate other viruses, such as COVID-19.

The manner in which system 1 of the current invention may operate is now further described. As shown, ring mount or ultrasound assembly 50 may be positioned at or near the top 20 of system 1 when the person steps into system 1. Where system 1 does not include enclosure 70, the person may simply step onto base 10. Where system 1 is enclosed, enclosure 70 may rotate to a position providing an opening for the person to enter. Once the person is standing on base 10, enclosure 70 may rotate to a closed position.

Once the person is in place, system 1 may be activated, through different mechanisms. For example, system 1 may be manually activated by an attendant who will control the flow of people from an outside location, through system 1, and into a designated or disinfected location. As another example, system 1 may include vacancy sensors which detect whether a person is positioned for disinfection. As another example, base 10 may include a sensor in active region 12 that senses the person's weight to activate system 1. As noted above, in addition to an attendant, or as an alternative thereto, software may be used to control the entry and departure of persons through system 1.

Regardless of how system 1 is manually or automatically activated, its activation preferably initiates the vertical movement of ring mount 50 to begin the disinfection scan of the person. In a preferred embodiment, ultrasound assembly 50 may start at top 20 and move vertically downward toward base 10. In some embodiments, this single scan may be adequate and the disinfected person may step out of system 1 and proceed to the disinfection area. In other embodiments, assembly 50 may perform a downward and upward scan on a person. Whether a single or double scan (or some other multiple of scans) is/are performed, it is preferred that, by the end of the scanning procedure, ultrasonic generators 50 have exposed all the person's skin and clothing to ultrasonic waves for inactivation of any pathogens present on them. Ultrasonic waves 54 may be generated on one or both of the downward and upward movements of assembly 50, depending on the intensity, speed and distances involved, and the level of disinfection desired.

If the disinfection involves only a downward scan, ring mount 50 may then be raised to top position 20 at which it started, and start at top 20 for the next person. Alternatively, assembly 50 may remain at or near base 10 when beginning the next scan.

After the disinfection procedure, or where higher levels of pathogen inactivation or desired such as sterilization, has concluded, the person just exposed to ultrasonic waves may step out of the system, and the process may be repeated for the next person.

Additional embodiments of the current invention are now described with reference to FIG. 6.

As shown in FIG. 6, mount or ring 50 may include one or more sources 53 of air that direct the air in the same general direction as the ultrasonic waves, and towards the person in disinfection zone 5. Though air is described herein, other fluids may be used. As an example, sources 53 may comprise air nozzles 53. Air nozzles 53 may be spaced in between ultrasound transducers 52 as shown and/or at different spacing.

Air nozzles 53 may provide high velocity air traveling in the same or similar direction as the ultrasonic waves. In this manner, the high velocity air may be coupled with the ultrasonic waves, thereby providing a medium in which the ultrasonic waves may travel. This in turn may facilitate delivery of the ultrasonic waves at the desired intensity and distance, e.g., intensity and distance required to reach the potential source of contamination, e.g., the person's skin or clothing, and disinfect it. As with other parameters, software may be used to control the intensity at which the air is emitted from nozzle 53.

In one embodiment, the present invention involves an air-coupled-ultrasound based disinfection system comprising an ultrasound generating system and an operator software, to manually adjust the ultrasound intensity output by the ultrasound generating system, and to manually adjust duration of ultrasound exposure to achieve the level of disinfection desired. The operator software may set the system on autorun at a preset input setting and auto-occupancy and vacancy sensors to initiate or stop the disinfection procedure and a base, i.e., space for a user (person to be disinfected) to occupy during the disinfection process.

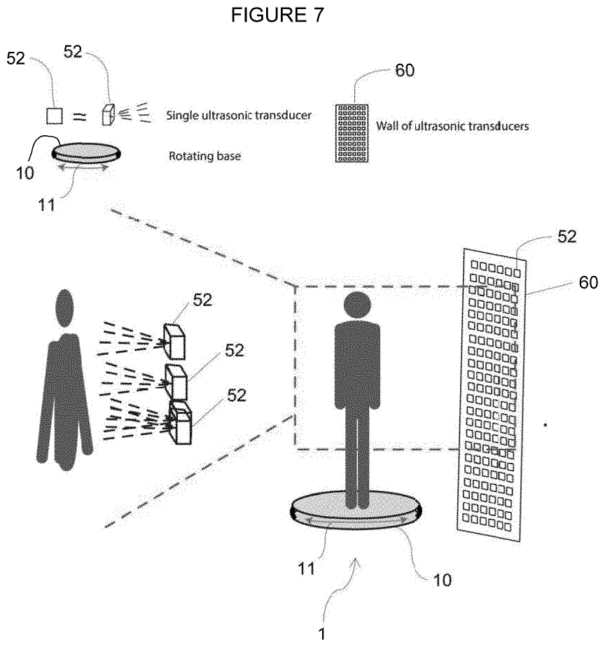

Additional embodiments of the current invention are now described with reference to FIGS. 7-9.

As shown in the embodiment of FIG. 7, ultrasound sources 52 may be located on a stationary mount or wall 60, which may be perpendicular to a stationary base 10 that a user can occupy. In general, ultrasound sources 52, as mounted to wall 60, may form an ultrasound array that provides an area of ultrasound coverage and intensity that may be varied as desired. In order to expose the person to be disinfected more completely, base 10 may physically rotate in a clockwise or counterclockwise direction 11 relative to wall 60 and ultrasound transducer array. The speed at which base 10 rotates may vary, but for example, it may rotate in a slow motion to treat all exposed and potentially contaminated fomites and skin of the person to be disinfected. As with other parameters discussed above, the speed and direction of rotation 11 may be controlled by software.

As alternative with this embodiment, the base 10 need not rotate and the person may be instructed to have one scan, and then turn around for another scan. Any number of scans may be taken in this manner.

In the embodiment shown in FIG. 7, the source of ultrasound 60 may be located on a stationary mount perpendicular to a stationary base, and may be coupled with a motor-operated rotating base 10 capable of rotating in the clockwise or anticlockwise direction 11 at a preset speed to facilitate exposure of all exposed and potentially contaminated fomites and skin. As an alternative, the preset speed may be varied by software and/or according to conditions present.

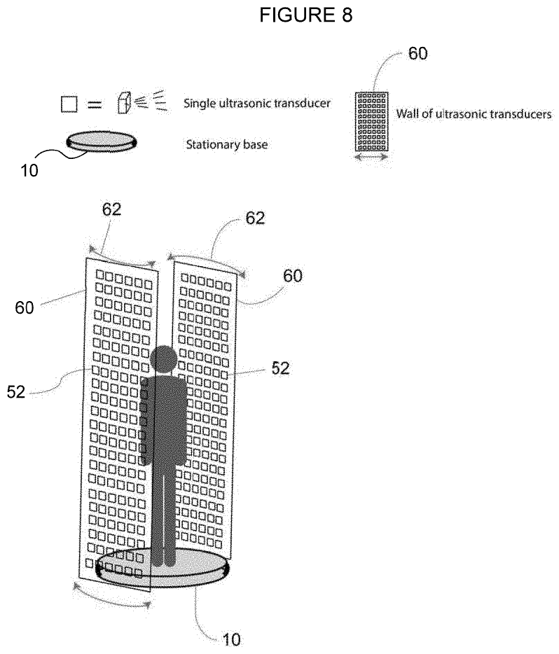

In another embodiment as shown in FIG. 8, two parallel mounts or walls 60 carrying the sources 52 of ultrasonic waves may be situated perpendicularly to a circular or elliptical base 10 with the base being situated between the ultrasound mounts 60. In this embodiment, when a user stands on the 10 base, one mount is in front of the user (person to be disinfected) and one behind the user and the mounts 60 are connected to motor systems (not shown) within the unit and the motors facilitate the mounts or walls 60 to move in a semicircular motion 62 along the two opposing halves of the circular or elliptical base 10. Employing two mounts 60 may lessen the disinfection time and could be utilized in very high traffic public locations.

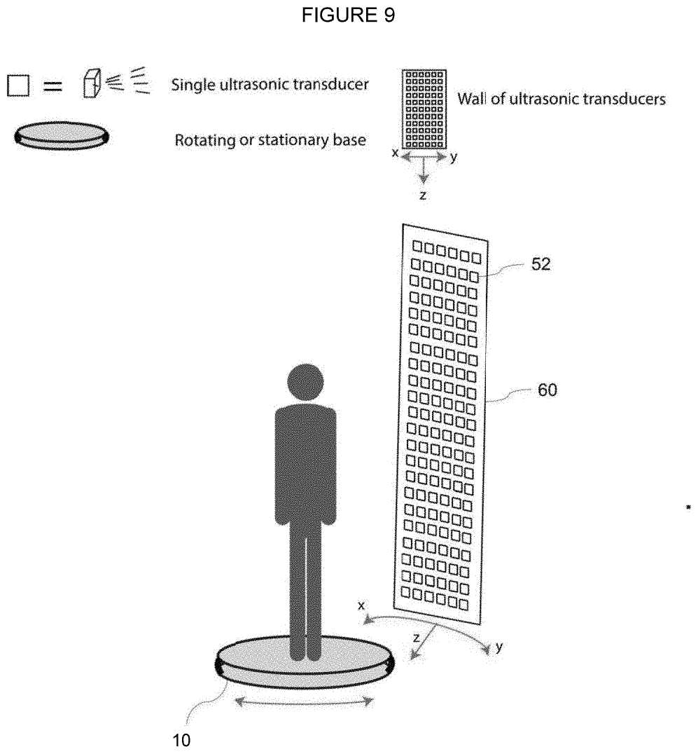

In yet another embodiment as shown in FIG. 9, the mount or wall 60 including the ultrasound sources 52, may be equipped with infrared sensors and a base and motor-enabled motion allowing the mount or wall 60 to not only move in x and y directions, i.e., along the shape of the base 10, but also allowing motion in the z direction, i.e., towards the occupant (person to be disinfected). Motion in the z direction may be guided by the infrared sensors to avoid physical contact between the mount or to maintain a preset distance between the mount and the occupant. Movement in the z direction can aid in reducing the distance between the ultrasound source and the person utilizing the system. Motion in z direction may be aided by infrared sensors as they can detect the location of the occupant. The embodiment of FIG. 9 may be coupled with a rotating or stationary base 10.

The system of the current invention has innovative applications. For example, the system may be used at an entrance to a public event, e.g., concerts, where many people would be in close proximity to each other; and where current social distancing rules would be difficult to enforce. In this situation, the system or a number of systems could be installed at entrances to public spaces for quick disinfection of transferable contaminants (i.e., harmful pathogens) traveling via a person's skin or clothing. The system of the present invention may also be used as part of the security check at airports, to avoid the situation where pathogens may be easily transferred from one person to another within the enclosed environment of the plane.

While the invention has been described in connection with what is presently considered to be the most practical and preferred embodiments, it is to be understood that the invention is not to be limited to the disclosed embodiment, but on the contrary, is intended to cover various modifications and equivalent arrangements included within the spirit and scope of the appended claims.

What is claimed is:

1. A non-contact ultrasound disinfection system, comprising:
a space for a person to be positioned within the system;
one or more ultrasound sources that are located in a non-contact position in proximity to the person;
an assembly that is coupled to the one or more ultrasound sources and that moves the one or more ultrasound sources along the person while the one or more ultrasound sources emit ultrasonic waves at the person.

2. The non-contact ultrasound disinfection system of claim 1, wherein the assembly comprises a ring that is configured to surround the person as it moves along the person, and that is coupled to a plurality of ultrasound sources.

3. The non-contact ultrasound disinfection system of claim 2, wherein the ring positions the plurality of ultrasound sources so that they emit the ultrasonic waves inward towards the person.

4. The non-contact ultrasound disinfection system of claim 1, further comprising a drive that is coupled to the assembly and that moves the assembly so that the one or more ultrasound sources move along the person.

5. The non-contact ultrasound disinfection system of claim 4, wherein the drive includes a motor and a jack screw.

6. The non-contact ultrasound disinfection system of claim 4, wherein the drive comprises a motor and pulleys and cables.

7. The non-contact ultrasound disinfection system of claim 1, further comprising:
a base; and
one or more struts that extend upward from the base and that is coupled to the assembly.

8. The non-contact ultrasound disinfection system of claim 4, further comprising an enclosure that encloses the person during a disinfection procedure.

9. The non-contact ultrasound disinfection system of claim 1, wherein the one or more ultrasound sources deliver an ultrasound dosage to the person to inactivate pathogens existing on the person's skin and/or clothing.

10. The non-contact ultrasound disinfection system of claim 1, wherein the one or more ultrasound sources are positioned a distance from the person so that the ultrasonic waves retain sufficient intensity to inactivate pathogens existing on the person's skin and/or clothing.

11. An apparatus for decontamination of contaminants traveling via a person's skin or clothing, comprising:
a disinfection space for a person to stand within;
one or more ultrasonic wave generators, each functioning to decontaminate pathogens present on the person's skin or clothing; and
one or more air nozzles to aid ultrasonic waves reach the target distance while maintaining sufficient intensity to inactivate pathogens.

12. The apparatus of claim 11, further comprising computer software to enable communication between the apparatus and an operator, thereby allowing the operator to initiate the present deactivation cycle or adjust the level of disinfection required, including altering the rate or power of ultrasonic wave exposure.

13. The apparatus of claim 11 may be operated manually via a computer software and screen or may be equipped with an automated occupancy sensor to initiate the preset decontamination cycle upon detecting presence of an individual.

14. The apparatus of claim 1 may further comprise of occupancy sensors shaped as feet to guide a person to occupy the appropriate location within the unit, such that the unit only initiates decontamination when/if the occupant is properly located within the unit.

15. The apparatus of claim 1 may additionally be equipped with a vacancy sensor to abort the present decontamination cycle if the person occupying the unit exits the unit or vacates the appropriate location within the unit i.e. the guiding sensors.

16. The apparatus of claim 1 further comprises of a power source to operate the motor and/or the ultrasonic transducers.

17. The apparatus of claim 1 may be equipped with a source to provide air to the air nozzles.

18. The apparatus of claim 1 further comprises indicators showing whether the unit is in use or vacant and ready for the next user.
